(19) United States
(12) Patent Application Publication
(10) Pub. No.: US 2025/0332323 A1
(43) Pub. Date: Oct. 30, 2025

(54) TISSUE MATRICES INCORPORATING MULTIPLE TISSUE TYPES

(71) Applicant: LifeCell Corporation, North Chicago, IL (US)

(72) Inventors: Sangwook Park, Virginia Beach, VA (US); Hui Xu, Plainsboro, NJ (US); Aaron M. Barere, Hoboken, NJ (US); Israel James Jessop, Garden Ridge, TX (US); Mrinal Shah, Parsippany, NJ (US)

(21) Appl. No.: 18/971,287

(22) Filed: Dec. 6, 2024

Related U.S. Application Data
(63) Continuation of application No. 18/783,906, filed on Jul. 25, 2024, now abandoned.
Application No. 18/783,906 is a continuation of application No. 18/117,782, filed on Mar. 6, 2023, now abandoned.
Application No. 18/117,782 is a continuation of application No. 16/838,512, filed on Apr. 2, 2020, now abandoned.
Application No. 16/838,512 is a continuation of application No. 15/639,592, filed on Jun. 30, 2017, now Pat. No. 10,639,398.
(60) Provisional application No. 62/358,347, filed on Jul. 5, 2016.

(51) Int. Cl. A61L 27/36; A61F 2/12; A61L 27/48; A61L 27/56; A61L 27/60

(52) U.S. Cl. CPC A61L 27/3645; A61F 2/12; A61L 27/3604; A61L 27/362; A61L 27/3633; A61L 27/48; A61L 27/56; A61L 27/60

(57) ABSTRACT

The present disclosure provides tissue products produced from extracellular tissue matrices. The tissue products can include acellular extracellular matrices including combinations of different tissue types. The combination can harness various properties of the different tissues to provide improved composite structures with desired mechanical and/or biologic properties.

This application is a continuation of U.S. patent application Ser. No. 18/783,906, which was filed on Jul. 25, 2024, which is a continuation of U.S. patent application Ser. No. 18/117,782, which was filed on Mar. 6, 2023, which is a continuation of U.S. patent application Ser. No. 16/838,512, which was filed on Apr. 2, 2020, which is a continuation of U.S. patent application Ser. No. 15/639,592, filed on Jun. 30, 2017, now U.S. Pat. No. 10,639,398, which claims priority under 35 U.S.C. § 119 to U.S. Provisional Application No. 62/358,347, filed on Jul. 5, 2016. The entire contents of each of the above-referenced applications are incorporated herein by reference in its entirety.

### BACKGROUND

The present disclosure relates to tissue products, and more particularly, to tissue matrices produced from a combination of two or more different tissue types.

Various tissue matrix products (e.g., acellular tissue matrices or similar tissue-derived or tissue regenerative materials) are currently available. Such products can be used to regenerate, reinforce, replace, and/or augment existing tissues, or tissues damaged or lost due to disease, trauma, surgery, radiation, or other events. Such materials can be very effective for treatment of many conditions. For example, acellular tissue matrix products such as ALLODERM®, an acellular human dermal matrix, and STRATTICE™, an acellular porcine dermal matrix (both from LIFECELL® CORPORATION, BRANCHBURG, NEW JERSEY), are useful for many surgical procedures, including abdominal wall defect repair and breast reconstruction.

Although currently available acellular tissue matrix products can be very effective at regenerating a range of tissue types, there remains a need for tissue matrix products that harness the beneficial regenerative and structural properties of tissue products derived from multiple tissue types. Accordingly, the present disclosure provides improved tissue matrix products that include combinations of two or more tissue matrix materials (i.e., materials derived from two or more types of tissues). The tissue matrix materials are arranged to provide improved methods of treatment—in some case, taking advantage of the biologic and mechanical properties of each of the component materials.

According to one embodiment, a tissue product is provided. The product can include a first component comprising an intact acellular tissue matrix and a second component comprising a porous acellular tissue matrix sponge covering at least a portion of the intact acellular tissue matrix. The porous acellular tissue matrix sponge comprises a tissue matrix that has been mechanically homogenized, resuspended, and stabilized, and wherein the intact acellular tissue matrix and porous acellular tissue matrix sponge are attached such that the intact acellular tissue matrix provides mechanical support to the porous acellular tissue matrix sponge.

According to one embodiment, a tissue product is provided. The product can include a first component comprising a sheet of acellular tissue matrix, and a second component comprising a porous acellular tissue matrix sponge covering at least a portion of the intact acellular tissue matrix. The second component may consist essentially of adipose tissue matrix.

According to one embodiment, a tissue product is provided. The tissue product can include a first component comprising a sheet of acellular tissue matrix and a second component comprising a sheet of a second acellular tissue matrix derived from a tissue type different than that of the first component. The product can further comprise a third component including a porous acellular tissue matrix sponge, wherein the third component is contained between the first component and the second component.

### DESCRIPTION OF THE DRAWINGS

FIG. 1A is a side cut away view of a tissue product including tissue matrix from two or more tissue types, according to various embodiments.

FIG. 1B is a side cut away view of a tissue product including tissue matrix from two or more tissue types, according to various embodiments.

FIG. 1C is a side cut away view of a tissue product including tissue matrix from two or more tissue types, according to various embodiments.

FIG. 1D is a side cut away view of a tissue product including tissue matrix from two or more tissue types, according to various embodiments.

FIG. 2A is a perspective view of the tissue product of FIG. 1B.

FIG. 2B is a perspective view of the tissue product of FIG. 1C.

FIG. 3 is a side cut away view of a tissue product including tissue matrix from two or more tissue types, according to various embodiments.

FIG. 4 is a perspective view of another tissue product including a sheet of acellular tissue matrix and a porous tissue matrix sponge covering opposing sides of the tissue matrix.

FIG. 5 is a perspective view of another tissue product including a sheet of acellular tissue matrix and a porous tissue matrix sponge covering a side of the tissue matrix.

FIG. 6 is a perspective view of another tissue product including two sheets of acellular tissue matrix and a porous tissue matrix sponge secured between the sheets.

FIG. 7 is a side cut away view of another tissue product including two sheets of acellular tissue matrix and a porous tissue matrix sponge secured between the sheets, the tissue product forming a volume shaped for implantation within a breast.

FIG. 8 illustrates the tissue product of FIG. 7 implanted within a breast to facilitate a breast augmentation, reconstruction, or other breast procedure.

FIG. 9 is a perspective view of another tissue product including two sheets of acellular tissue matrix and a porous tissue matrix sponge secured between the sheets.

FIG. 10 illustrates a side cut away view of tissue products within a breast to facilitate an improved breast procedure using products and methods of the present disclosure.

FIG. 11 is a perspective view of another tissue product including a sheet of acellular tissue matrix and a porous tissue matrix sponge.

### DESCRIPTION OF CERTAIN EXEMPLARY EMBODIMENTS

Reference will now be made in detail to certain exemplary embodiments according to the present disclosure, certain examples of which are illustrated in the accompanying drawings. Wherever possible, the same reference numbers will be used throughout the drawings to refer to the same or like parts.

In this disclosure, the use of the singular includes the plural unless specifically stated otherwise. In this disclosure, the use of “or” means “and/or” unless stated otherwise. Furthermore, the use of the term “including”, as well as other forms, such as “includes” and “included”, is not limiting. Any range described herein will be understood to include the endpoints and all values between the endpoints.

The section headings used herein are for organizational purposes only and are not to be construed as limiting the subject matter described. All documents, or portions of documents, cited in this disclosure, including but not limited to patents, patent applications, articles, books, and treatises, are hereby expressly incorporated by reference in their entirety for any purpose.

Various human and animal tissues can be used to produce products for treating patients. For example, various tissue products for regeneration, repair, augmentation, reinforcement, and/or treatment of human tissues that have been damaged or lost due to various diseases and/or structural damage (e.g., from trauma, surgery, atrophy, and/or long-term wear and degeneration) have been produced. Such products can include, for example, acellular tissue matrices, tissue allografts or xenografts, and/or reconstituted tissues (i.e., at least partially decellularized tissues that have been seeded with cells to produce viable materials).

A variety of tissue products have been produced for treating soft and hard tissues. For example, ALLODERM® and STRATTICE™ (LIFECELL CORPORATION, Branchburg, NJ) are two dermal acellular tissue matrices made from human and porcine dermis, respectively. Although such materials are very useful for treating certain types of conditions, materials having different biological and/or mechanical properties may be desirable for certain applications. For example, ALLODERM® and STRATTICE™ have been used to assist in treatment of structural defects and/or to provide support to tissues (e.g., for abdominal walls or in breast reconstruction), and their strength and biological properties make them well-suited for such uses.

Such materials, however, may not be ideal for regeneration, repair, replacement, and/or augmentation of certain soft-tissue defects. For example, improved tissue fillers for replacing lost or damaged tissues, including adipose or other soft tissues in the form of porous sponges, may be beneficial for some patients. Such porous sponges, however, may lack sufficient structural integrity for certain uses.

For example, tissue matrix sponges may have insufficient tensile strength, burst strength, or suture retention strength to provide needed support in various procedures such as breast reconstruction, breast augmentation, abdominal wall defect treatment, or treatment of soft tissues that are subjected to repeated movements, or even occasionally experience relatively high mechanical stresses. In addition, some materials may have less than optimal compressive elasticity and strength, bending rigidity, kink resistance, or abrasion resistance. Accordingly, improved devices and methods are provided herein.

The improved devices incorporate combinations of tissue matrix sponges (e.g., adipose tissue matrix sponges) along with intact acellular tissue matrices, such as dermal, fascial, or muscle tissue matrices. The improved devices are joined to harness properties of the different tissue matrices, thereby providing improved regenerative biologic and mechanic properties for certain uses.

As used herein, the term “intact acellular tissue matrix” refers to an extracellular tissue matrix having a shape and form substantially similar to the tissue from which the matrix is derived, although it will be understood that production of the acellular matrix (e.g., by removing cells) will produce a matrix that is modified from the original tissue matrix, by for example, changing the microstructure of the matrix. For example, an “intact acellular tissue matrix” produced from elongated, sheet-like tissue such as dermis, bladder, intestinal layer(s), stomach layer(s), dura, pericardium, or fascia may be in the form of a sheet formed by the original protein structure. Such “intact acellular tissue matrices”, however, can include openings, meshes, or holes, as discussed further below, and may be modified, e.g., by cross-linking, enzymatic treatment, or chemical modification. “Intact acellular tissue matrices” would not include tissues that have been ground, cut, homogenized, or otherwise processed to form small tissue fragments or particles, even if such fragments or particles are resuspended or otherwise processed to produce a sheet or other form.

Accordingly, in various embodiments, tissue products for treatment or regeneration of tissue are provided. The tissue products can include a first component comprising an intact acellular tissue matrix and a second component comprising a porous acellular tissue matrix sponge. The porous acellular tissue matrix may cover at least a portion of the intact acellular tissue matrix, or alternatively or additionally, the porous acellular tissue matrix can be positioned near the intact acellular tissue matrix during a surgical procedure. The porous acellular tissue matrix sponge can be produced in a variety of ways, as discussed further below.

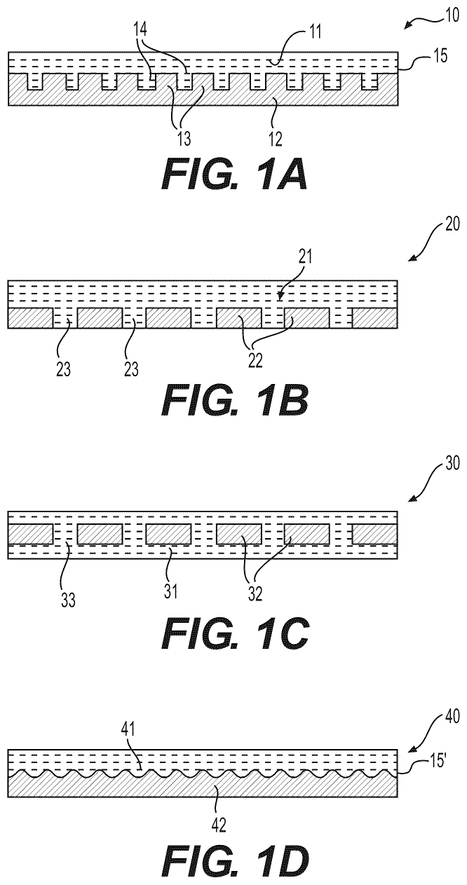

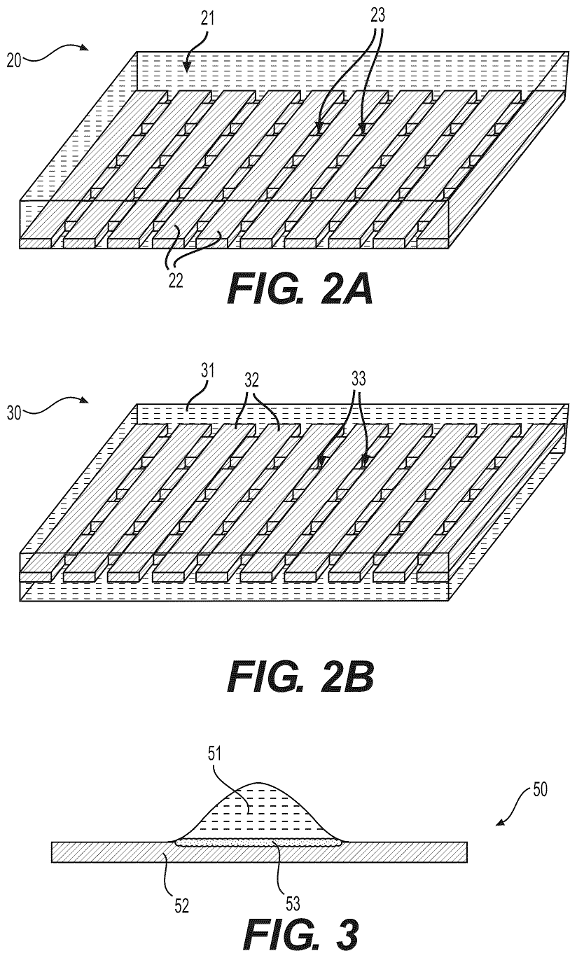

FIGS. 1A-11 illustrate tissue products or methods of using the tissue products of the present disclosure in different embodiments and configurations. FIGS. 1A-1D illustrate side cut away views of various tissue products, and FIGS. 2A-2B are perspective views of the devices of FIGS. 1B and 1C, respectively.

As shown, the products of FIGS. 1A-1D (10, 20, 30, 40) can include multiple components. As discussed above, the products can include a first component (12, 22, 32, 42) and a second component (11, 21, 31, 41). The first component (12, 22, 32, 42) can include an intact acellular tissue matrix, and the second component (11, 21, 31, 41) can include a porous tissue matrix sponge, both of which will be described in more detail.

The first component (12, 22, 32, 42) can include a variety of suitable acellular tissue matrices in the form of a sheet, or other suitable three-dimensional structure, including for example, folded shapes, boxes, cup-like shapes, tubes, irregular or repeating patterned shapes, spheres or other rounded 3-D shapes. The intact tissue matrix (first component) can be formed from a variety of different tissue sources and can be processed and configured to provide structure support or mechanical stability to the devices (10, 20, 30, 40).

The tissue matrices used to produce the first component (12, 22, 32, 42) can include a variety of suitable tissue matrix materials. Examples of the tissues that may be used to construct the tissue matrices for the first component can include, but are not limited to, skin, parts of skin (e.g., dermis), fascia, muscle (striated, smooth, or cardiac), pericardial tissue, dura, umbilical cord tissue, placental tissue, cardiac valve tissue, ligament tissue, tendon tissue, blood vessel tissue, such as arterial and venous tissue, cartilage, bone, neural connective tissue, urinary bladder tissue, ureter tissue, and intestinal tissue. For example, a number of biological scaffold materials that may be used for the first component are described by Badylak et al., Badylak et al., “Extracellular Matrix as a Biological Scaffold Material: Structure and Function,” Acta Biomaterialia (2008), doi:10.1016/j.actbio.2008.09.013. In some cases, the first component includes a sheet of acellular tissue matrix derived from human or porcine dermis. Suitable human and porcine dermal materials include, for example, ALLODERM® and STRATTICE™, respectively.

The first component (12, 22, 32, 42) can be selected based on both biologic and mechanical properties. For example, suitable tissue matrix materials used to produce the first component will generally be capable of providing a regenerative tissue scaffold suitable for supporting the ingrowth of native cells and formation of tissue without excessive inflammation and with minimal scar formation.

In addition, the first component (12, 22, 32, 42) can be selected based on its ability to provide a desired amount of structural support. For example, the first component (12, 22, 32, 42) may possess sufficient tensile strength, burst strength, and suture retention strength to allow use of the device for treatment of such conditions as complex or simple abdominal wall defects, defects associated with surgical oncology for treatment of breast cancer, treatment of structural defects in connective tissues (e.g., fascia, tendons, or ligaments), and/or to provide structural support around breast implants or tissue expanders used in augmentation or reconstruction.

As noted above, however, the devices can include a second component (11, 21, 31, 41), and the second component can be selected to provide specific biologic and mechanical properties. For example, in one embodiment, the second component includes a tissue matrix derived from adipose tissue. The tissue matrix derived from adipose tissue can be selected based on its ability to support regeneration of certain tissue types, including regeneration of adipose tissue, or predominantly adipose tissue; and may be selected to produce a desired feel, compressibility, size, shape, or other mechanical features. As such, the second component can support a desired level of adipose regeneration, which may be desirable for various anatomic sites, including, for example, the breast (e.g., after surgical removal of tumors or for augmentation), the buttocks, thighs, neck, face, or any other site where adipose tissue may be desirable to produce a certain feel, cosmetic appearance, biologic property, or other intended result.

The first component (12, 22, 32, 42) and second component (11, 21, 31, 41) can be arranged in a variety of ways to produce desired mechanical and biologic properties when implanted in vivo. For example, FIGS. 1A-1D illustrate various configurations of devices (10, 20, 30, 40) consistent with the present invention.

As shown in FIG. 1A, the first component 12 can be formed as a sheet of intact acellular tissue (e.g., acellular dermal matrix), and the second component 11 can be formed on one side of the first component 12 to produce a bilayer sheet or composite structure (e.g., having a sheet of the first component 12 with a mass of the second component 11 as a sheet or other configuration). In one embodiment, the device 10 can optionally include a textured surface 15 on at least one side of the sheet of first component 12 to assist in mechanical attachment of the first component 12 and second component 11.

The textured surface 15 can facilitate joining between the first component 12 and the tissue fibers in a slurry or suspension used for form the second component 11 (discussed below). The textures can be grooves or holes, or can include a roughened surface such that it creates jagged edges or textures with some loose collagen fibers that facilitate cross-linking. The textured surface 15 can include a surface roughening (e.g., formed by scraping or other mechanical process). In addition, the textured surface 15 can include indentations 14 and/or protrusions 13 that allow interdigitaion of the first and second components. Alternatively, as shown in FIG. 1D, the textured surface 15′ can include small irregularities or changes in the surface shape for the first component 42 and or second component 41.

Furthermore, although the embodiments described above with respect to FIGS. 1A, 1B, and 1D illustrate a device having the second component attached to or covering one side of a sheet of the first component, it will be appreciated that the second component may be applied to both sides (e.g., the top and bottom) of a sheet of the first component, or may be applied only to a portion of one or both of the top and bottom surfaces of the first component.

In other embodiments, the first component can be in the form of a sheet having openings or a mesh structure in which the second component can be positioned. For example, FIGS. 1B and 1C illustrate devices 20, 30 having a first component 22, 32 in the form of a sheet with openings 23, 33, and including a second component 21, 31 covering one or both sides of the sheet of first component 22, 32, while filing the openings 22, 32. Such configurations are illustrated in more detail in FIGS. 2A and 2B, which provide perspective views of the devices of FIGS. 1B and 1C, respectively.

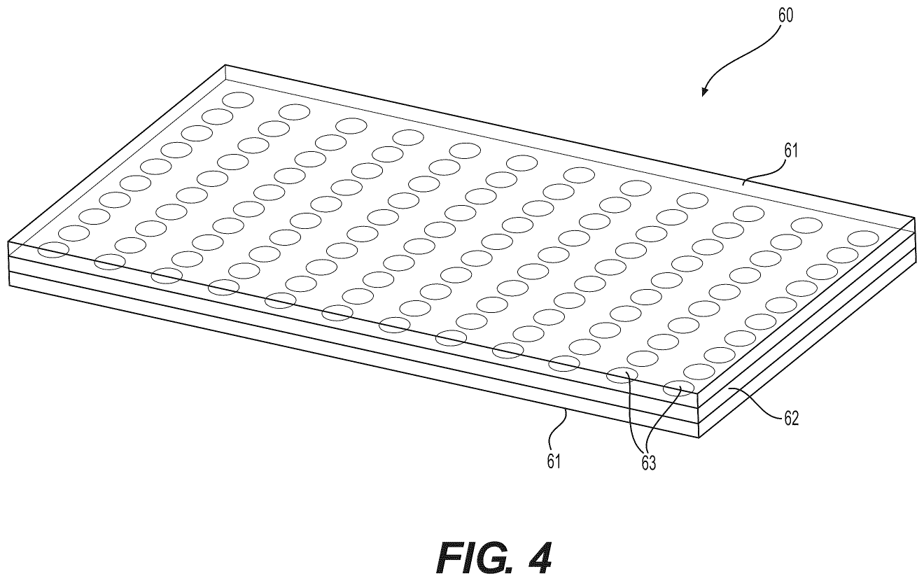

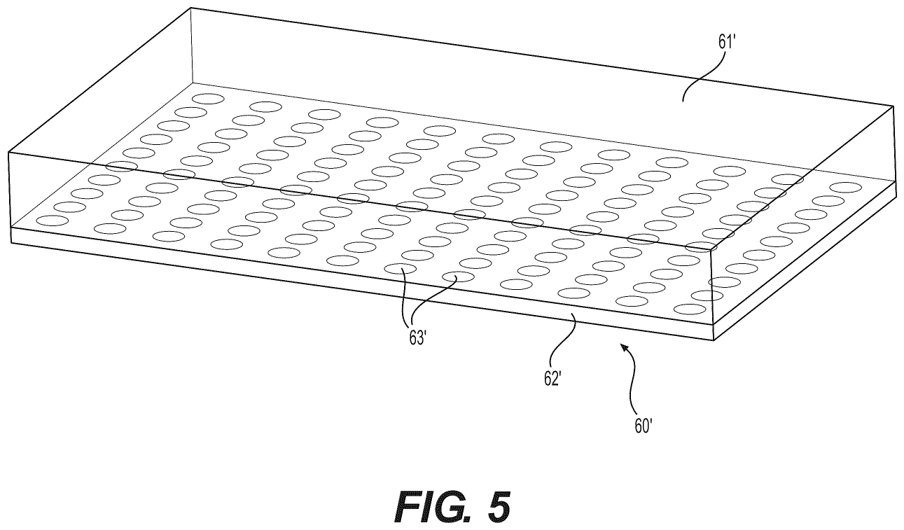

It should be noted, that the pattern of openings or meshwork in the devices of FIGS. 1A and 1B can be varied based on a number of factors. For example, FIGS. 4 and 5 are perspective views of tissue products including a sheet acellular tissue matrix and a porous tissue matrix sponge covering one side or both opposing sides of the tissue matrix. As shown, FIG. 4 illustrates a device 60 including a sheet of the first component (an intact acellular tissue matrix) 62 with openings 63 formed as circular holes. In addition, the device 60 includes a second component 61 covering an opposing side of the sheet of the first component 62 and filling the openings 63. Similarly, the device 60′ of FIG. 5 includes a sheet of the first component (an intact acellular tissue matrix) 62′ with openings 63′ formed as circular holes. In addition, the device 60′ includes a second component 61′ covering one side of the sheet of the first component 62′ and filling the openings 63′.

The devices illustrated in FIGS. 4-5 (as well as those in FIGS. 1A-2B) can be used for breast reconstruction applications or other treatments. The size of the openings 63 can be selected based on the desired strength, wherein the size of the openings 63 and spacing of openings can be varied to produce desired properties. The size and number of the openings can be selected to modulate the rate of cellular ingrowth into the openings 63 (or corresponding parts of FIGS. 1A-2B). In addition, the second component 61 can be formed of a material that is more suitable for cellular ingrowth, and may provide a better biological response while harnessing the mechanical properties of the first component 62.

The devices illustrated in FIGS. 4-5 (as well as those in FIGS. 1A-2B) can be designed for specific applications. The designs may depend on the specifics of the application. For example, the design in an application where strength needs to be maintained, shear is low, and fluid flow across the intact matrix is not important (e.g., facial reconstruction or minimally invasive facelifts) can include smaller openings 63. Conversely, when more compressibility is important, larger and more openings 63 might be better.

In some embodiments, the second component may cover or be applied to only a portion of a surface of the first component, and/or may be applied as a bulk material having a desired shape. For example, FIG. 3 is a side cut away view of another tissue product, according to various embodiments. As shown the device 50 of FIG. 3 includes a sheet of the first component 52, with the second component 51 covering a limited section 53 of the first component, but it should be understood that the second component may cover more or different sections. The section 53 may have a modified surface (e.g., by roughening or texturing) that facilitates stronger bonding between the second component 51. As such, the second component can be in the form of a bulky or larger mass, and the device 50 may be used, for example, in breast surgery, wherein the first component 52 provides structural support, e.g., to attach to the chest wall or muscle, and the second component 51 provides an improved biological response to allow rapid ingrowth to produce tissue with a desired composition, texture, feel, and biological properties.

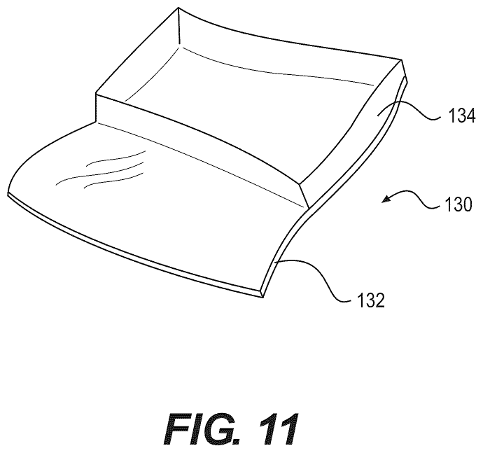

Similar to the device of FIG. 3, FIG. 11 is a perspective view of another tissue product 130 including a sheet 132 of intact acellular tissue matrix and a porous tissue matrix sponge 134. FIG. 11 is intended to illustrate that the sponge 134 (corresponding to the second component 51 of FIG. 3) can include a variety of configurations (e.g., covering differing amounts of the sheet 132). The product 130 can, like the product of FIG. 3, be implanted to treat a breast, but as with FIG. 3's embodiment, could also be used to treat other sites (e.g., a facial defect, buttock, abdominal wall, or other structure). Furthermore, the components of the product 130 can be provided as a single device or as separate components to facilitate methods of treatment discussed further below.

The devices and methods described herein can also include more than two types of tissue matrices. Specifically, in some cases, the devices include a first component comprising a sheet of acellular tissue matrix and a second component comprising a sheet of a second acellular tissue matrix derived from a tissue type different than that of the first component. In addition, the devices can include a third component comprising a porous acellular tissue matrix sponge, wherein the third component is contained between the first component and the second component. Embodiments including such structures as well as their uses and methods of use are discussed further below.

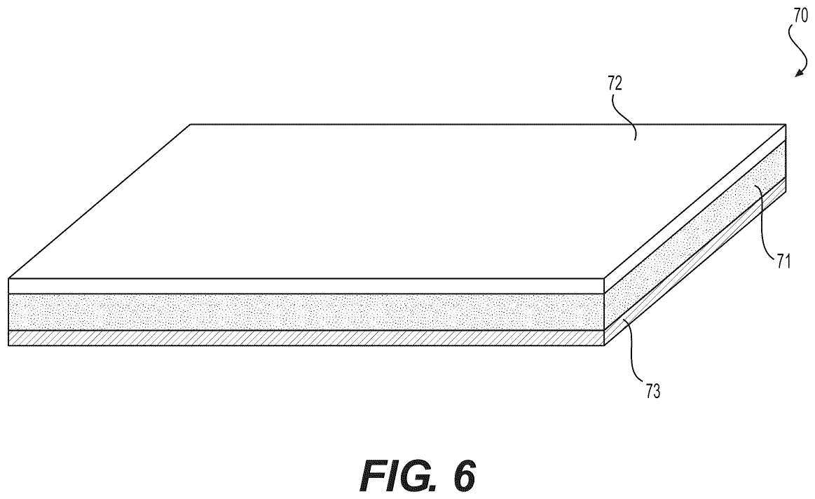

FIG. 6 is a perspective view of another tissue product 70 including two sheets 72, 73 of acellular tissue matrix and a porous tissue matrix sponge 71 secured between the sheets 72, 73. As shown, the device 70 is provided in the form of flat or flexible sheet of material, but may include a variety of different shapes, including a box-like shape. Alternatively, the device 70 can include any suitable three-dimensional form such as a spherical, ovoid, or irregular three-dimensional form.

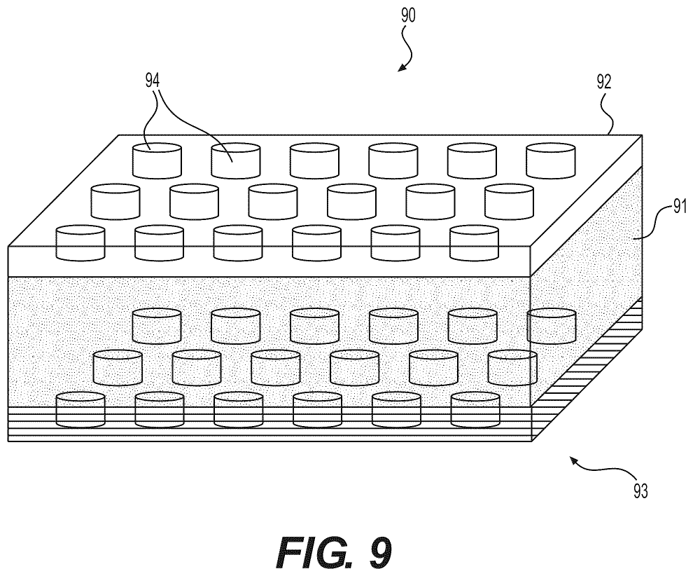

Furthermore, the configuration of FIG. 6 can be modified to include openings in the sheets 72, 73. For example, FIG. 9 illustrates an embodiment of a device 90, including sheets 92, 93, similar to those of FIG. 6, as well as a sponge 91, but further includes openings or perforations. The openings or perforations may include a variety of sizes, shapes, spacings, or configurations, as discussed above with respect to FIGS. 4 and 5.

In some cases, the device 70 of FIGS. 6 can be modified to produce a material shaped for a specific use, e.g., as a breast implant. Suitable breast implants may be used, for example, for augmentation or reconstruction after procedures such as mastectomy, skin-sparing mastectomy, lumpectomy, or any other procedure such as revision breast augmentation, breast augmentation, or mastopexy.

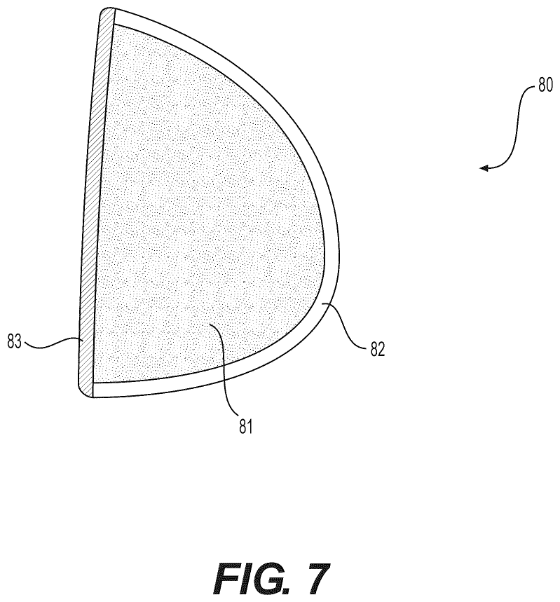

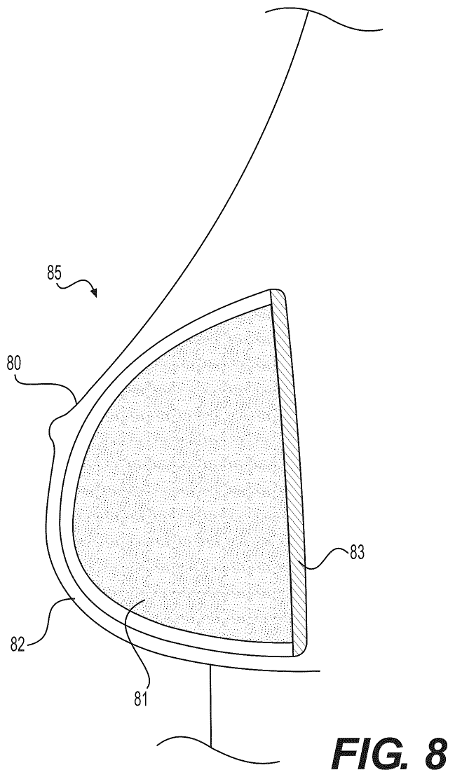

FIG. 7 is a side view of another tissue product 80, for use as a breast implant, and FIG. 8 illustrates implantation of the device 80 within a breast 85. As shown, the device 80 includes including two sheets 82, 83 of acellular tissue matrix and a porous tissue matrix sponge 81 secured between the sheets to form a volume shaped for implantation within a breast.

The device 80 is illustrated having the shape of a typical breast implant, such as a rounded or teardrop implant. The devices 80, however, of the present disclosure need not have typical breast implant (teardrop or rounded) shapes. For example, the devices can have other shapes including, for example, irregular shapes, spherical shapes, ovoid shapes, or custom-made shapes based on patient anatomy or treatment site. For example, a surgeon may select a spherical or custom-made shape for implantation in a lumpectomy site or based on patient-specific factors. In addition, the surgeon may select two-or more implants to be implanted next to one another or in different locations. In addition, although described in particular with respect to breast implants the presenting disclosed implants, systems, and methods can be used at other sites where synthetic implants may be used (e.g., gluteal implants).

As discussed above, the two sheets 82, 83 can include intact acellular tissue matrix, but can be formed from tissue matrix derived from different tissue types. For example, in one embodiment, the first sheet 82, which may form an anterior or frontal portion of the implant 80 can be formed from a tissue matrix selected to allow cellular ingrowth and tissue regeneration, while also providing sufficient mechanical properties (e.g., tensile strength, burst strength, suture retention strength) to allow the sheet 82 to provide structural support and load-bearing capacity, as may be needed to support a mass of the tissue matrix sponge 81 and surrounding breast structures. In some embodiments, the sheet 82 is a dermal acellular tissue matrix such as ALLODERM® or STRATTICE™. The sheet can include other tissue such as bladder, intestinal layer(s), stomach layer(s), dura, pericardium, skeletal muscle, nerve, peritoneum, or fascia.

The second sheet 83 can be produced from a different tissue. For example, one suitable tissue can include a muscle tissue matrix. Suitable muscle matrix materials are described in U.S. Patent Publication Number 2015/0282925A1 (application Ser. No. 14/410,204), which was filed on Jul. 1, 2013 to Xu et al.

As shown in FIG. 8, the device 80 can be implanted within the breast 85 with the first sheet 82 facing substantially anteriorly, and the second sheet 83 facing posteriorly and near or in contact with chest wall muscles (e.g., the pectoralis muscles). As such, the first sheet 82 can be configured to allow cellular ingrowth and tissue revascularization from cutaneous or immediately subcutenous tissues, or from anteriorly located tissue within a pocket formed in a breast 85. In addition, the second sheet 83 can allow cellular ingrowth from posteriorly located (relative to the implant 80) tissues such as muscle, and may support desired muscle generation.

As discussed above, the devices described herein in each of the figures can include acellular tissue matrix sponge (11, 21, 31, 41, 51, 61, 71, 81). As used herein, tissue matrix sponge will be understood to refer to a tissue matrix material that has been processed to produce a sponge-like matrix. The sponge-like matrix can be formed of an extracellular tissue matrix (ECM) that retains ECM components including extracellular collagen, glycoproteins, and other molecules important for supporting cellular ingrowth and tissue regeneration.

As used herein, the term “tissue matrix sponge” will be understood to refer to a tissue matrix material that includes extracellular tissue matrix (ECM) (including collagen and important non-collagenous proteins), wherein the ECM has been mechanically processed to form fragments or particles, and has been resuspended or reformed (e.g., by casting and drying) to form a porous sponge-like material. The sponge properties can be tailored by selecting an appropriate tissue type (e.g., using adipose, dermis, muscle, fascia, nerve, vascular tissue, intestinal components, skeletal muscle, peritoneum, tendon, ligament, or other appropriate tissue).

Further, the tissue properties can be modified by controlling solid content of the sponge (i.e., the about of tissue matrix present in suspension). Higher solid content will generally form higher modulus and stronger materials. Additional modifications to the material to adjust mechanical properties can include chemical cross linking, particle or fiber sizes, increasing solid content per volume through compression, and pattern molding with fiber alignment for directional properties.

The tissue matrix sponge can be formed from a number of tissue matrix types and with a number of processes. For example, in some embodiments, the tissue matrix sponge is formed from adipose tissue. Suitable adipose tissues are described generally is number U.S. Patent Publication Number 2012/0310367A1 (application Ser. No. U.S. 13/483,674, filed May 30, 2012, to Connor). Such adipose materials can be formed generally by mechanical homogenization, washing, resuspension, and stabilization of the material. The material may be dried (e.g. by freeze drying before or after stabilization), and stabilization can be by dehydrothermal treatment (DHT), cross-linking (UV, radiation, or chemical cross-linking). The stabilization can further be used to bond or attach the sponge to the other material. For example, DHT treatment can cause some degree of cross-linking, which can be improved by surface roughening to texturing (see e.g., FIG. 3, item 53). In addition, the sponge may be sterilized before or after joining to the intact tissue matrix. Sterilization may be performed after the components of the devices described herein are joined. Further, the sponge may be formed while in contact with the intact acellular tissue matrix components, or may be formed separately prior to joining.

In addition, although the devices shown above have a number of layers or components, it will be appreciate that the structures can include additional layers. For example, devices including multiple layers of the components shown in the figures may be used. And multiple layers of the tissue matrix components can be added, for example, to improve device strength.

As noted above, the products discussed herein can be used for treatment of breast. And, in varying embodiments, the intact acellular tissue matrix component and porous tissue matrix sponge can be provided either as a single premanufactured article, or as separate components to be implanted by a surgeon, or as combinations of single articles and separate components (e.g., for different section of a treatment site).

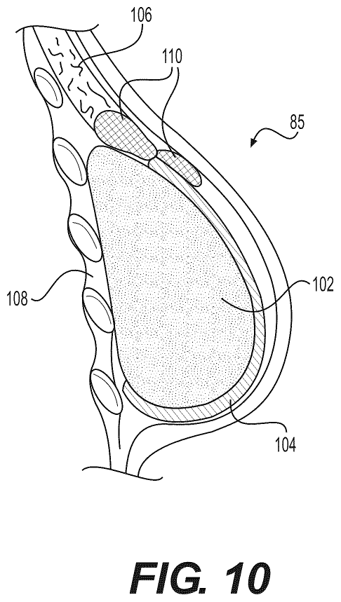

For example, FIG. 10 illustrates a side cut away view of a prospective use of the tissue product of FIG. 7 implanted, together with additional tissue products, within a breast to facilitate an improved breast procedure using products and methods of the present disclosure.

As shown, the products of the present disclosure can be implanted at a breast 85 treatment site, e.g., for augmentation or reconstructive procedures. As such, the products can be used in conjunction with various implants 102 or similar devices (e.g., tissue expanders) and can be used for a variety of implant procedures and location (e.g., subcutaneous or subpectoral). And the products of the present disclosure, including an intact acellular tissue matrix sheet 104 can be implanted to support the implant or tissue expander and/or to facilitate other goals (e.g., provide tissue coverage, restore blood flow, etc.

In some breast treatment procedures, however, in addition to the benefits provided by the intact tissue matrix sheets, it is desirable to implant other tissue, such as adipose tissue matrix sponge, to provide additional implant coverage. For example, in some cases, e.g., with thin patients, the implant may cause undesirable effects such as skin rippling or less than desirable shapes (e.g., due to the bulk of the implant at the superior portion or other areas with insufficient breast tissue coverage).

Accordingly, in various embodiments, a surgeon may use a sheet of tissue matrix 104 and a tissue matrix sponge such as adipose matrix 110. The adipose matrix can be provided as a separate component and implanted where needed, or can be provided preassembled and attached to the sheet of tissue matrix (as shown in FIG. 11). In the latter case, the sponge 134 can be provided with sufficient bulk so that the surgeon can shape or contour (e.g., remove portions) of the sponge to produce a desired implant.

The sponge 110 or 134 can be implanted at a variety of sites around the breast to prevent rippling, reduce undesired shapes (e.g., reduce appearance of the superior portion of the implant as illustrated in FIG. 10), and/or to improve overall aesthetic results.

The above description and embodiments are exemplary only and should not be construed as limiting the intent and scope of the invention.

1. A tissue product, comprising:
a first component comprising a porous acellular tissue matrix sponge comprising a tissue matrix that has been mechanically homogenized, resuspended, and stabilized;
a second component comprising an intact acellular tissue matrix covering at least a first portion of the porous acellular tissue matrix sponge; and
a third component comprising an intact acellular tissue matrix covering at least a second portion of the porous acellular tissue matrix sponge opposite the first portion.

2. The tissue product of claim 1, wherein the second component comprises an intact dermal acellular tissue matrix.

3. The tissue product of claim 1, wherein the third component comprises an intact dermal acellular tissue matrix.

4. The tissue product of claim 1, wherein the tissue product is in the form of a sheet.

5. The tissue product of claim 1, wherein the tissue product is in the form of a sphere, ovoid, or irregular three-dimensional form.

6. The tissue product of claim 1, further comprising one or more perforations.

7. The tissue product of claim 6, wherein the one or more perforations are situated in the second component.

8. The tissue product of claim 6, wherein the one or more perforations are situated in the third component.

9. The tissue product of claim 6, wherein the one or more perforations are circular.

10. The tissue product of claim 6, wherein the first component fills the one or more perforations.

11. The tissue product of claim 6, wherein the one or more perforations are sized to modulate the rate of cellular ingrowth into the one or more perforations.

12. The tissue product of claim 1, wherein the second component comprises an intact muscle acellular tissue matrix.

13. The tissue product of claim 1, wherein the third component comprises an intact muscle acellular tissue matrix.

14. The tissue product of claim 1, wherein the first component comprises muscle acellular tissue matrix.

15. The tissue product of claim 1, wherein the first component comprises adipose acellular tissue matrix.

16. A method of treatment comprising;
selecting a tissue product, wherein the tissue product comprises;
a first component comprising a porous acellular tissue matrix sponge comprising a tissue matrix that has been mechanically homogenized, resuspended, and stabilized;
a second component comprising an intact acellular tissue matrix covering at least a first portion of the porous acellular tissue matrix sponge; and
a third component comprising an intact acellular tissue matrix covering at least a second portion of the porous acellular tissue matrix sponge opposite the first portion; and
implanting the tissue product into a patient.

17. The method of claim 16, wherein implanting the tissue product comprises implanting the tissue product subcutaneously.

18. The method of claim 16, wherein implanting the tissue product comprises implanting the tissue product subpectorally.

19. The method of claim 16, wherein implanting the tissue product comprises placing the tissue product over a breast implant or tissue expander.

20. The method of claim 16, wherein implanting the tissue product comprises affixing the tissue product to the chest wall, muscle, or skin of the patient.
